(19) United States
(12) Patent Application Publication
(10) Pub. No.: US 2025/0044283 A1
(43) Pub. Date: Feb. 6, 2025

(54) MONITORING COVID-19 PROGRESSION AND TREATMENT

(71) Applicant: SEROXO LIMITED, Surrey (GB)

(72) Inventors: David SARPHIE, Surrey (GB); Rubina Mian, Birmingham (GB)

(73) Assignee: SEROXO LIMITED, Surrey (GB)

(21) Appl. No.: 18/275,343

(22) PCT Filed: Feb. 1, 2022
(86) PCT No.: PCT/GB2022/050262

§ 371 Date: Aug. 1, 2023

Related U.S. Application Data
(60) Provisional application No. 63/144,613, filed on Feb. 2, 2021.
(60) Provisional application No. 63/192,232, filed on May 24, 2021.

(51) Int. Cl. G01N 33/50; G01N 21/76

(52) U.S. Cl. CPC G01N 33/5091; G01N 21/76; G01N 2333/165; G01N 2800/26

(57) ABSTRACT

The present invention provides a rapid method using whole blood samples for determining the severity of COVID-19 disease arising from SARS-CoV-2 infection and monitoring progression and treatment of the disease relying on determination of the functionality of leukocytes (predominately neutrophils) to exhibit challenge-induced superoxide anion production with quantification by chemiluminescent measurement.

### FIELD OF THE INVENTION

The present invention relates to in vitro methods for monitoring progression and treatment in individuals suffering COVID-19 disease arising from SARS-CoV-2 viral infection. More particularly, methods are provided which rely on determination of the responsiveness of leukocytes (predominately neutrophils) to exhibit challenge-induced superoxide anion production, i.e. produce a “respiratory burst” in response to in vitro activation. Whilst such activation of leukocytes is known and is often referred to as leukocyte coping capacity (LCC), the invention provides new application in the field of COVID-19 disease monitoring and more generally in monitoring progression and treatment of disease associated with viral infection liable to cause acute respiratory distress syndrome (ARDS). This may be disease associated with another coronavirus which causes severe acute respiratory distress syndrome and hence categorised as a SARS virus or any other known or future emerging virus which causes similar serious illness in humans.

It has been shown that hospitalised patients with severe COVID-19 disease exhibit highly significant differences in measured challenge-induced superoxide anion (also known as “reactive oxygen species” or ROS) production in whole blood samples compared to healthy individuals similarly tested. Such a test is thus now proposed as a rapid and highly convenient means of assessing status of COVID-19 disease in patients with SARS-CoV-2 infection or status of any other viral-caused ARDS, more particularly status warranting hospitalisation or hospitalisation and further treatment. Such a test is also proposed as means of gaining quick and convenient guidance on effectiveness of therapeutic treatment of such disease.

### BACKGROUND TO THE INVENTION

Chemical inducers such as phorbol myristate acetate (PMA) are well-known for activating neutrophils in peripheral blood samples whereby retained capacity for superoxide production can be quantified as a measure of neutrophil functionality (Hu et al. Cell Signal (1999) 11, 335-360). Such use with whole blood samples with measurement of superoxide production by chemiluminescence to obtain an LCC score forms the basis of a test commercialised by Oxford MediStress Limited to quantify psychological stress in humans and animals; see European Patent no. 1558929 and related patents deriving from published International Application WO2004/042395. Psychological stress depresses retained neutrophil functionality and thereby LCC score as determined by such a test. As indicated above, this same test has now been applied to whole blood samples from patients with severe Covid-19 disease with the finding that such patients exhibit far higher LCC scores than healthy individuals similarly tested.

It is known that SARS-CoV-2 infection triggers a multi system inflammatory disorder which can lead to a spectrum of clinical symptoms. (Dhama et al. 2020). However, the clinical symptoms of patients with Covid-19 vary widely. About half of individuals infected are asymptomatic or mildly symptomatic showing typical clinical signs of the common cold such as fever, muscle ache, cough, shortness of breath and fatigue (Feng et al. 2020; Tabata et al 2020).

The difference between the development of moderate and severe disease appears to occur roughly after 10 days. The current criteria for classifying mild cases and severe cases are mainly based on respiratory rate, oxygen saturation and the ratio of arterial oxygen partial pressure (PaO₂ in mmHg) to fractional inspired oxygen (FiO₂) expressed as a fraction PaO₂/FiO₂. Whilst helpful, these measures give no indication of impending intracellular dysfunction and the extent of the multi-system inflammatory processes.

Severely ill patients maintain a sustained profile of high pro-inflammatory cytokines, referred to as a cytokine storm (Lucas et al. 2020). These include high levels of interleukins, TNF-α, G-CSF, MCP-1 and MIP-1a, which are higher in intensive care unit patients than non-intensive care unit patients (Lucas et al. 2020; Huang et al. 2020; Liu et al 2020). Some patients go on to develop acute respiratory distress syndrome (ARDS), pulmonary oedema and multiple organ dysfunction syndrome (MODS), leading to a high mortality (Li et al. 2020). Approximately 20% of patients display acute multi-system failure, including ARDS, accompanied by an intense inflammatory process, which is life-threatening (Wang et al. 2020; Wu et al. 2020; Yang et al. 2020).

The unpredictable nature of the disease results in patients with mild symptoms suddenly progressing to ARDS, septic shock or MODS (Chen et al. 2020). Once the disease has progressed to severe or critical illness it is difficult to resolve (Li et al. 2020) and prognosis is poor. What is clearly needed is the ability to monitor the inflammatory state of the individual to determine if individual treatments and interventions are working and to optimise individuals based on the objective cellular status of the cells.

One of the most life-threatening complications associated with severe COVID-19 and multi-system inflammation is the appearance of blood clots in the arteries, veins and microscopic vessels. These occur in approximately half of all patients with severe symptoms and have been shown to trigger strokes and impair oxygen exchange by restricting blood flow in the lungs. However, exactly what causes these clots to appear is unknown. Severe COVID-19 is associated with a higher neutrophil to lymphocyte ratio in infected tissues and high levels of pro-inflammatory cytokines. It has been suggested that neutrophils could be contributing to an extensive micro-thrombus formation resulting in multi-organ failure (Cavalcante-Silva et al. 2020). However, exactly how they could be contributing to this is unknown.

Neutrophils are the most abundant immune cells in human blood. They account for approximately 50-70% of all leukocytes and have been described as the protagonists of inflammation (Soehnlein et al. 2017). As both the first line of innate defence and effectors of adaptive immunity, polymorphonuclear leukocytes (PMNs), including neutrophils, play crucial roles in the immune defence against bacterial, fungal and viral infections (Mocsai, 2013; Kruger et al., 2015). They produce inducible reactive oxygen species, with superoxide anion as the primary product, via the NADPH oxidase complex (Panday et al. 2015; Nguyen et al 2017; Lambeth, 2004; Mocsai, 2013) from two sources, one pool of ROS being produced by the membrane complex and the other situated intracellularly within the neutrophil (Karlson-Bengtsson et al. 2000). It was speculated that these two different pools have different functional roles.

Reactive oxygen species are not simply purveyors of damage but can modulate a host of cellular and systematic functions ranging from cell homeostasis to cell death. Molecular actions include both inhibition and activation of proteins, mutagenesis of DNA and activation of gene transcription. Cellular actions include promotion or suppression of inflammation, immunity and carcinogenesis (Nathan & Cunningham-Bussel, 2013).

ROS can also activate surrounding PMNs by initiating the release of granules, inducing the generation of neutrophil extracellular traps (NETs) and stimulating the production of the pro-inflammatory cytokines such as tumour necrosis factor alpha (TNFα) and macrophage inflammatory protein 2 (MIP-2) (Brinkmann et al., 2010; Naik and Dixit, 2011; Sheshachalam et al., 2014). In fact, these downstream effects of ROS production may ultimately be responsible for much of the activities of ROS rather than direct damage by ROS themselves (Miralda et al., 2017).

A brisk immune response can clear a pathogen but can cause extensive tissue damage and neurodegeneration. When uncontrolled it can provoke severe inflammatory reactions. Consequently, the activation of oxidative burst must be tightly regulated and checkpoints exist to restrict the times and locations that are appropriate for cellular functions (Nathan and Cunningham-Bussel, 2013). However, little is known of the production of ROS in Covid-19.

Patients with less severe COVID-19 often have near normal or slightly low numbers of peripheral white blood cells and lymphopenia. However, in severe COVID-19, the lymphocytes count decreases progressively, while the neutrophils count gradually increases (Li et al. 2020). To date, four meta-analyses have reported that patients with severe COVID-19 infection have a higher NLR (Neutrophil/Lymphocyte ratio) than those with non-severe Covid-19 infection (Zheng et al. 2020, Chan & Rout. 2020; Ghahramani et al. 2020; Lagunas-Rangel et al. 2020). There is no absolute reported objective cut off point of when NLR progresses from near normal through moderate to severe. An objective marker of cellular dysfunction would be a helpful tool for the clinician in monitoring changes to the patient status and to determine if interventions are having positive effect.

The NLR ratio has been considered an independent risk factor for mortality in hospitalised patients (Cavalcante-Silva et al. 2020). Neutrophils operate using a number of different mechanisms including chemotaxis, phagocytosis, release of ROS, and granular proteins and the production and liberation of cytokines (Selder et al. 2017; Hellebrekers et al. 2018). Studies have shown that neutrophils can release elevated NETs (neutrophil extracellular traps) in response to COVID-19. NETs can act as a double-edged sword of immunity, having a pro- or anti-inflammatory effect (Cavalcante-Silva et al. 2020).

Neutrophils also interact with other immune cells, releasing cytokines; degranulation produces an oxidative burst and NETs (Galani & Andreakos 2015: Naumenko et al. 2018). The increase in NLR is often accompanied by an increase in D-dimer and C-reactive protein (CRP) (Ponti et al. 2020; Ye et al. 2020).

Lung autopsies of deceased patients have revealed neutrophil infiltration in pulmonary capillaries, their extravasation into the alveolar spaces and neutrophilic mucositis (Laforge et al. 2020; Fox et al. 2020; Yao et al. 2020; Barnes et al. 2020). Wang et al (2020) also demonstrated that neutrophilia coincides with lung injury in severe COVID-19 patients. Increased levels of circulating neutrophil extracellular traps (NETs), which are indicative of neutrophil activation, have also been described in patients (Golonka et al. 2020).

The high neutrophil to lymphocyte ratio observed in critically ill patients with COVID-19 is associated with excessive levels of ROS, which promote a cascade of biological events that drive pathological host responses. ROS induces tissue damage, thrombosis and red blood cell dysfunction, which can contribute to COVID-19 disease severity (Laforge et al. 2020). Neutrophils also play the lead role in thrombotic complications associated with COVID-19 (Petito et al. 2020).

Respiratory burst from activated neutrophils resulting in superoxide anions and H₂O₂, leads to oxidative stress that contributes to the cytokine storm and, as indicated above, is implicated in blood clot formation in SARS-CoV-2 infection (Cecchini & Cecchini. 2020; Laforge et al. 2020).

As discussed above, neutrophils also have an important role in the modulation of the immune system in the innate and adaptive immune response. Newly identified human neutrophil subsets can suppress T-cell activation and proliferation (Mortaz et al. 2020) and their presence may provide a pivotal role in the immune response to COVID-19.

Moreover, immature phenotype and/or dysfunctional mature neutrophils have been reported in severe COVID-19 patients. These studies indicate that the increased infiltration of immature and/or dysfunctional neutrophils contributes to the imbalance of the lungs' immune response in severe cases (Schulte-Schrepping et al. 2020; Parackova et al. 2020).

It is notable that Zheng et al. in their 2020 paper noted above pose the question “Can we predict the severity of coronavirus disease 2019 with a routine blood test?” but do not provide any answer on how this might be achieved in a physiologically relevant manner. They and others have merely suggested focussing on the neutrophil-to-lymphocyte (NLR) ratio in blood samples. This ignores that neutrophils, as will be evident from the above discussion, are subject to complex environmental regulation of an enzymic system as regards ROS production (see again Panday et al. 2015 and Nguyen et al. 2017). Counting of neutrophil numbers is not equatable with neutrophil functional activity and even less cannot predict superoxide anion induced production from neutrophils in an in vitro blood sample. Despite the immense amount of studies providing evidence of a dysregulated in vivo innate immune response in some COVID-19 patients, it was only with the studies reported herein that it became apparent that measurement of retained functional capacity of leukocytes (primarily neutrophils) to produce ROS in response to PMA in whole blood samples can provide a rapid 10 minute detection method for objectively determining onset or occurrence of severe COVID-19 and monitoring progression and treatment of COVID-19.

As noted above, upon activation, NADPH oxidase of neutrophils catalyses the transfer of electrons from NADPH to molecular oxygen generating superoxide anions (O₂⁻) as the primary product. Superoxide anions can give rise to other reactive oxygen species, e.g. hydroxyl radicals (Ngyugen et al. 2017) but in the context of an in vitro test of the invention, it will be appreciated that detection of produced ROS can be a measure of induced superoxide anion and as such these may be referred to interchangeably.

Status of COVID-19 patients is commonly scored with reference to the WHO recognised Ordinal Scale for Clinical Improvement as set out immediately below. See also WHO R &D Blueprint novel Coronavirus COVID-19 Therapeutic Trial Synopsis (February 2020).

### Ordinal Scale for Clinical Improvement

| Patient State | Descriptor | Score |
|---|---|---|
| Uninfected | No clinical or virological | 0 |
| | evidence of infection | |
| Ambulatory | No limitation of activities | 1 |
| | Limitation of activities | 2 |
| Hospitalised | Hospitalised, no oxygen | 3 |
| Mild disease | therapy | |
| | Oxygen by mask or nasal | 4 |
| | prongs | |
| Hospitalised | Non-invasive ventilation or | 5 |
| Severe disease | high-flow oxygen | |
| | Intubation and mechanical | 6 |
| | ventilation | |
| | Ventilation + additional | 7 |
| | organ support - pressors, | |
| | RRT, ECMO | |
| Dead | Death | 8 |

By severe COVID-19 disease as used herein will be understood disease warranting at least hospitalisation or hospitalisation and provision of oxygen, i.e. at least 3 or 4 on the above scale, e.g. severity consistent with requirement for surveillance and treatment in an intensive care unit (ICU) which may equate with a score exceeding 4.

### SUMMARY OF THE INVENTION

Although the invention is principally discussed herein with reference to COVID-19, it will be understood that it is of more general clinical applicability. The present invention thus provides a method of assessing disease progression in a subject suspected or known to have a viral infection capable of causing acute respiratory disease syndrome (ARDS), e.g. more particularly, for example, suspected or known to have COVID-19 disease arising from SARS-CoV-2 infection, which comprises:
(a) contacting a whole blood sample obtained from said subject with an inducer capable of stimulating superoxide production in neutrophils under conditions suitable for such stimulation;
(b) determining the increase of superoxide production above basal in said test sample after a time period to obtain a first result and
(c) comparing said first result with a second comparator result, wherein said second comparator result is derived from carrying out steps (a) and (b) with blood samples from healthy individuals or is a pre-determined threshold which correlates with one or more criteria equating with onset or occurrence of an ARDS disease status,
   whereby ARDS disease status is determined.

Such disease status may accord with desirability for hospitalisation or desirability for hospitalisation and provision of a specific treatment, e.g. provision of oxygen supply.

Thus where the patient is suspected or known to have COVID-19, in step (c) said first result may be compared with a second comparator result representing a pre-determined threshold for onset or occurrence of severity of at least 3 (warranting at least hospitalisation), or at least 4 (warranting provision of oxygen) or at least 5 (warranting non-invasive ventilation or high flow oxygen) or at least 6 (warranting intubation and mechanical ventilation) on the WHO Ordinal Scale for Clinical Improvement. It may accord with severity meriting care in an ICU, generally at least 4 or at least 5 on the same scale.

The invention also provides a method of monitoring treatment in a subject known to have disease arising from a viral infection capable of causing ARDS, more particularly for example COVID-19, which comprises carrying out steps (a) and (b) as above and in step (c) comparing said first result with a second comparator result which has been taken from the subject at an earlier time point at the start or during treatment, e.g. when a COVID-19 patient has symptoms consistent with at least a score of any of 3 to 6 on the WHO Ordinal scale as noted above, whereby reduction in induced superoxide production in the test sample compared with in said second comparator sample is indicative of reduction in disease severity. Such reduction in disease severity will be consistent with observed reduction in neutrophil functionality level. Such steps may be repeated thereby providing a highly convenient means for long-term (“longitudinal”) monitoring of the effect of treatment on the patient's physiological status. Such methodology is envisaged as a convenient means for example for aiding decision-making by clinicians in applying treatment for COVID-19 disease.

Whilst herein the invention is described primarily with reference to determining progression of disease and monitoring treatment in patients infected with SARS-CoV-2, as indicated above, it will be appreciated that the invention is more widely applicable to viral infections capable of causing ARDS, such as other coronaviruses, e.g. SARS viruses, known or emerging in the future with the capability of causing severe acute respiratory syndrome or other potentially pandemic-causing viruses which cause serious lower respiratory tract illness in humans.

Generally, chemiluminescence detection of superoxide production will be employed as noted above.

Commonly, where superoxide production is determined after a short period of inducer challenge by conventional chemiluminescence measurement, e.g. employing luminol and a portable luminometer, the basal chemiluminescence invariably will be so low in samples as not to require consideration. Thus in these circumstances, total measured relative light units (RLUs) may be equated with induced superoxide production and as directly proportional to neutrophil functionality. This has been established to apply for example when using freeze-dried PMA/luminol reagent as supplied by Oxford MediStress for LCC scoring of whole blood samples and incubating the sample for 10 minutes at 37.5° C. in accordance with the standard protocol for use of that reagent. In other words, “determining the increase of superoxide production above basal” in the test sample may be equated with simple one step quantification of superoxide present at the end of the neutrophil stimulation period, e. g. 10-30 minutes after addition of the inducer.

Whilst the invention relies on assessing the capacity of the total leukocyte component of whole blood to produce superoxide in response to appropriate in vitro chemical challenge (commonly referred to as the leukocyte coping capacity (LCC) score), since this will largely be determined by neutrophil capability for such external stimulation, reference may alternatively be made to determining the individual's neutrophil functionality level.

In the context of the present invention, rather than LCC score it is deemed more appropriate to refer to LIT™ (Leukocyte ImmunoTest™) score. Fullerton et al. 2019 confirmed that an LCC test as discussed above specifically and rapidly (in 10 minutes) will quantify leukocyte (primarily neutrophil) ROS release using small whole blood samples from various sources. They showed that employing erythrocytes and plasma as substrate for such test generated negligible luminescence consistent with the PMA response being attributable to leukocytes

### BRIEF DESCRIPTION OF THE FIGURES

FIG. 1 illustrates use of LIT scores from a controlled clinical trial wherein LIT scores from patients clinically confirmed to have severe COVID-19 were compared with those from a healthy control group. The y-axis shows the mean values of the LIT measurements (triplicate sample measurements). The boxplot on the right-hand side shows the median and interquartile range of the scores for both groups. The dot plot on the left-hand side shows individual subject values and, as a thicker dot, the mean for the group. For both measures, the average LIT score for COVID-19 patients clearly exceeds the average for the healthy volunteer group.

FIG. 2: Box plots showing the distribution of LIT scores in COVID-19 patients depending on whether invasive ventilation was in use (Y) or not in use (N). The horizontal lines in each box represent the medians for each cohort.

FIG. 3: Comparison between LIT score v neutrophil count for the COVID-19 cohort and a sepsis cohort with LIT scores of those patients who died within 28 days circled. Broken circle: patient died within 24 hours of LIT test.

FIG. 4: Based on results obtained from COVID-19 patients (n=44), mathematical modelling (Linear Mixed Effects Model) was used to project the trajectory of PMNL (neutrophil count) from admission to ICU to discharge. There is no difference in PMNL neutrophil) trajectory between those patients who survived and those who died. The analysis shows that neutrophil count alone is not sufficient to make prediction of mortality risk.

FIG. 5: Using the same mathematical modelling as for FIG. 4, the Linear Mixed effects Model, applied to LIT/N results (results for LIT score divided by neutrophil count) for the same cohort of COVID-19 patients (n=44), the trajectory of LIT/N score from admission to ICU to discharge was assessed. This plot demonstrates that LIT/N scores in contrast to neutrophil count alone (FIG. 4) can provide a useful indicator of mortality risk.

### DETAILED DESCRIPTION

The samples employed for a method of the invention are whole blood samples and thus as indicated above superoxide production will strictly equate with leukocyte capacity for superoxide production (referred to as LCC score or LIT score). Such methodology importantly avoids centrifugation, which is known to affect cell reactivity, and also plating out of cells on glass slides which may also affect functionality.

It will be appreciated that a method of the invention maintains the three-dimensional structural integrity of leukocytes. The ability of leukocytes to produce reactive oxygen species is altered by cell signalling pathways of other entities (Mian et al. 2005). A method of the invention provides a physiologically relevant means for monitoring the cellular capacity of leukocytes to produce superoxide radicals in real time. The physiological relevance is convincing since leukocytes remain suspended in whole blood which permits dynamic three-dimensional interaction with surrounding hormones, cytokines, erythrocytes, platelets and cell-cell interaction within and between different leukocyte cohorts, NET's, interleukins and of course the SARS-CoV-2 virus. All have the potential to dramatically affect leukocyte responsiveness and expression of cell surface receptors. As cellular integrity is maintained and unadulterated, the cells are in a near physiological state; the potential disruption to cell signalling pathways is limited. Avoidance of cell centrifugation and plating out means the neutrophils are kept in a similar environment as they would be in vivo, surrounded by cells, mediators and hormones, all of which can influence their responsiveness. The ability of the neutrophils to produce ROS can be studied under near physiological surroundings. The ROS released can also activate surrounding PMNs by initiating the release of granules, inducing the generation of neutrophil extracellular traps (NETs), and stimulating the production of the pro-inflammatory cytokines such as tumour necrosis factor alpha (TNFα) and macrophage inflammatory protein 2 (MIP-2) (Brinkmann et al., 2010; Naik and Dixit, 2011; Sheshachalam et al., 2014). These mediators in turn can accentuate and enhance the production of ROS from surrounding neutrophils. Thus, compared to the traditional study of pure isolated neutrophils, the method of the invention enables rapid physiologically relevant assessment of function in neutrophils in whole blood from COVID-19 patients and the studies reported herein show for the first time that this can be used to provide a rapid objective method of judging severity of disease.

Blood samples as small as about 5-20 μl (preferably 10 μl) will suffice obtained, for example, using a conventional finger lancing device.

It may be chosen to additionally determine the leucocyte account, more preferably the neutrophil count, in blood samples to be tested in accordance with the invention. This may be conveniently expressed as neutrophils×10⁹/l. In some instances, if need be or desired, the measured superoxide production above basal for each sample may be corrected by reference to the number of leukocytes or neutrophils in the sample. Indeed, as shown by data herein it may be preferred to determine the challenge-induced increase of superoxide above basal per 10⁹ neutrophils/l, designated the LIT/N™ score.

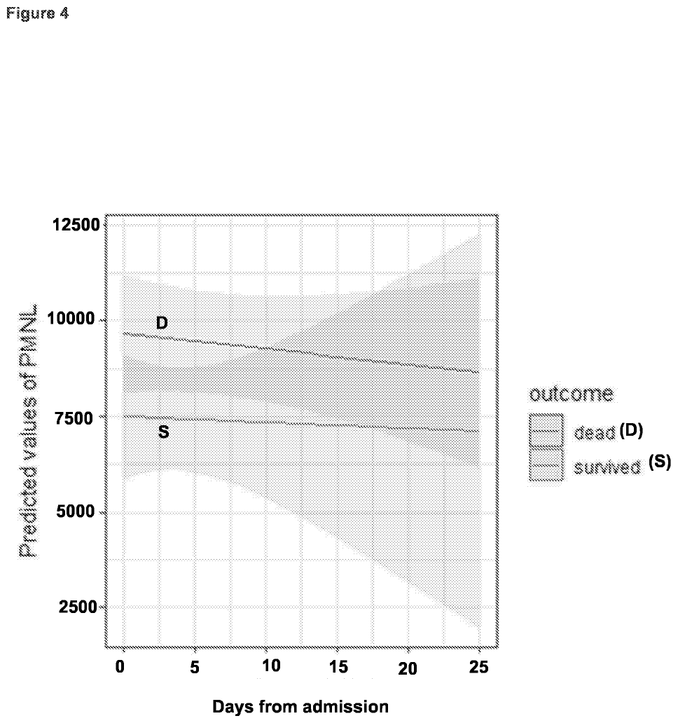

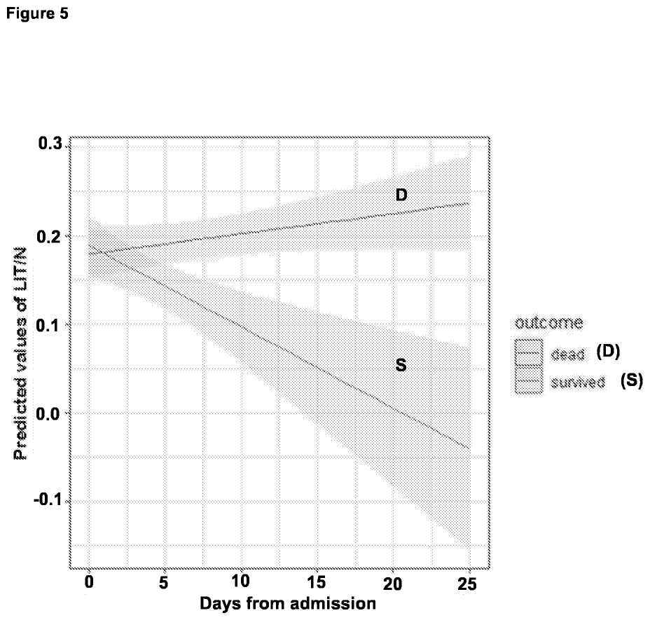

In particular, assessment of LIT/N scores at more than one time point, e.g. daily over two or more days, may be favoured, for example, in assessing mortality risk in patients with an acute respiratory disease syndrome arising from viral infection, especially for example, in assessing mortality risk in severe COVID-19 patients. Data now presented shows that in patients with COVID-19, a high LIT/N value as associated with COVID-19 patients with severe COVID-19 (at least 4 or 5 on the WHO Ordinal Scale, e.g. as assessed as requiring admittance to an ICU) which shows no downward trend with daily monitoring, is a useful indicator of mortality risk (see FIG. 5). In contrast, mere daily monitoring of neutrophil count in such patients has been found an insufficient indicator for this purpose (see FIG. 4).

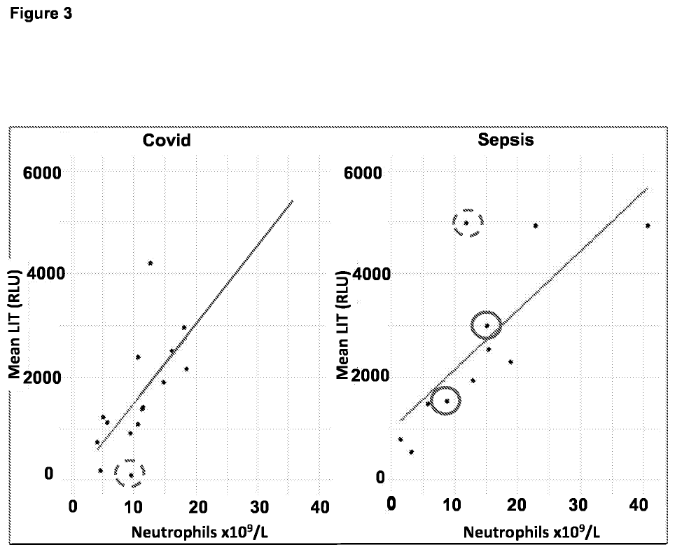

Alternatively, LIT and neutrophil count scores derived from a test blood sample may be compared with prior identically collected LIT and neutrophil count data from patients with the same disease, for example mapped to a LIT vs neutrophil count plot for the disease of concern, to determine the LIT-N™ status of the patient to be assessed. As illustrated by FIG. 3, this is proposed as an alternative means of assessing mortality risk, for example for COVID-19 patients.

As already noted above, superoxide production may be conveniently measured by known simple chemiluminescence measurement using, for example, luminol or iso-luminol. Suitable protocols are disclosed for example in European Patent no. 1558929 of Oxford MediStress. Generally, an incubation temperature of 37-37.5° C. will be chosen and incubation continued for a pre-determined time, preferably consistent with maximal or near maximal chemiluminescence measurement. As referred to above, the freeze-dried composition comprising PMA and luminol salt as supplied by Oxford MediStress for LCC testing enables suitable test results from a finger prick of whole blood to be attained in just 10 minutes and was used for the tests reported in Example 2 as a preferred reagent.

While a conventional luminometer may be employed for detection of the chemiluminescence, such light detectors require expensive and fragile photomultiplier tubes. Hence, use of an alternative photon detector may be preferred. In particular, a silicon photomultiplier (Si-PM) may, for example, be favoured. Such a photon detector is deemed more robust for the purpose and to combine greater cost-effectiveness with sufficient sensitivity of photon detection. A suitable hand-held luminometer is available again from Oxford MediStress as part of the CopingCapacity™ test kit, which also provides freeze-dried PMA/luminol-containing reagent composition as noted above.

A suitable photon detector may also be provided as a component of a mobile phone. Such a mobile luminometer for chemiluminescence detection has previously been proposed as part of a mobile chemistry platform (Roda et al. (2014) Anal. Chem. “Integrating Biochemiluminescence Detection on Smartphones; Mobile Chemistry Platform for Point-of-Need Analysis”) and may be similarly employed to enable carrying out methods of the invention even away from any surgery or hospital, for example in a home or on route to hospital.

Thus a method of the invention has all the following advantages for monitoring severity and treatment of COVID-19 disease:
Simple, easy to use-results obtainable in 10 minutes, minimally invasive.
Unit for protocol can be portable, not lab-based thereby minimising cost.
Allows integration of treatments based on objective assessment of clinically relevant neutrophil functionality.

Of particular relevance to convenience of use as noted above is that there is no necessity to fractionate blood samples to obtain an isolated leukocyte or neutrophil fraction. Blood samples for carrying out a method of the invention may be directly contacted with any chemical inducer capable of stimulating superoxide production in neutrophils. The inducer may be preferably phorbol myristate acetate (PMA), more particularly for example the microbial product phorbol 12-myristate 13-acetate obtainable from Sigma-Aldrich. However alternative inducers which might be employed are well-known. They include N-formyl-Met-Leu-Phe (fMLP chemotactic peptide), zymosan, lipopolysaccharide and adrenaline. The chemical inducer may be conveniently stored in the form of a freeze-dried reagent composition, e.g. as a pellet, for dissolution in an appropriate buffer solution, e.g. phosphate-buffered saline.

For additional convenience coupled with high sensitivity, as indicated above, luminol or iso-luminol may be conveniently supplied with the chemical inducer in a single reagent composition for addition to samples as exemplified by the commercially-available freeze-dried composition comprising PMA and luminol noted above.

In a further aspect of the invention, there is provided a system specifically configured for carrying out a method of the invention as discussed above, said system comprising a photon detector such as a portable luminometer for quantitative detection of chemiluminescence and a system for analysing the results and configured to provide an alert for a neutrophil functionality level associated with a pre-determined threshold correlating with a disease status, e.g. desirability of hospitalisation or hospitalisation and provision of oxygen or severity meriting patient monitoring and/or treatment in an ICU. The system may provide an alert of physiological status warranting change of a treatment.

The following non-limiting examples illustrate the invention.

### EXAMPLES

### Example 1: Example of Protocol for Use of PMA Challenge to Assess Neutrophil Functionality Level in Whole Blood Samples

To measure the background blood chemiluminescence level, 10 μl of whole blood is transferred into a silicon anti-reflective tube. 90 μl of 10⁻⁴ M luminol (5-amino-2, 3-dihydrophalazine-1,4-dione; Sigma-Aldrich) diluted in phosphate buffer is added. The tube is then shaken gently. To measure chemiluminescence produced in response to PMA challenge, 20 μl of PMA (Sigma-Aldrich) at a concentration of 10⁻³ M is added. For each tube, chemiluminescence may be measured for 30 secs every 5 mins in a luminometer, for a total of 30 mins. When not in the luminometer, tubes are incubated at 37.5° C., e.g. in a dry block heater.

A single reading after incubation at 37.5° C. for 10 minutes may be found convenient and more preferable.

It will be appreciated that the same challenge test may be carried out with any sufficiently sensitive photon detector, e.g. a Si-PM may be employed

### Example 2: Evaluation of LIT Scores for Patients with COVID-19

The study was approved by Birmingham University Ethical Committee and conducted at an NHS teaching hospital in Birmingham, UK. 15 patients in the intensive care unit (ICU) with a confirmed positive PCR test for SARS-CoV-2 and suffering severe COVID-19 were recruited to the study. Triplicate blood samples were obtained from each patient by venepuncture or finger prick (10 μl) and analysed for neutrophil functionality level using freeze-dried PMA/luminol reagent composition as commercially available from Oxford MediStress for LCC scoring. ROS production in response to PMA was analysed within 30 mins of taking blood. Triplicate LIT scores for neutrophil functionality level for each patient were averaged and the mean LIT score for each patient used for further analysis.

The collected data for each patient additionally included severity of COVID-19 (according to, WHO classification as noted above) age, sex, use of ventilation and type, lactate, co-morbidities, including diabetes, PaO₂/FiO₂ ratio, use of antibiotics, days in ICU, neutrophils and total leukocyte count. One patient had both COVID-19 and sepsis.

### Measurement of ROS Production by Luminometer (LIT Scoring)

A 10 μl blood sample was mixed with 100 μl phosphate buffered saline containing the freeze-dried PMA/luminol mixture. After incubation for 10 minutes at 37.5° C., the sample was evaluated for production of reactive oxygen species by measuring chemiluminescence in RLU (relative light units) using a portable luminometer (3M Clean-Trace®).

The test results from the “COVID” cohort were compared with results obtained from blood samples of a group of 18 healthy volunteers (“healthy volunteers” cohort).

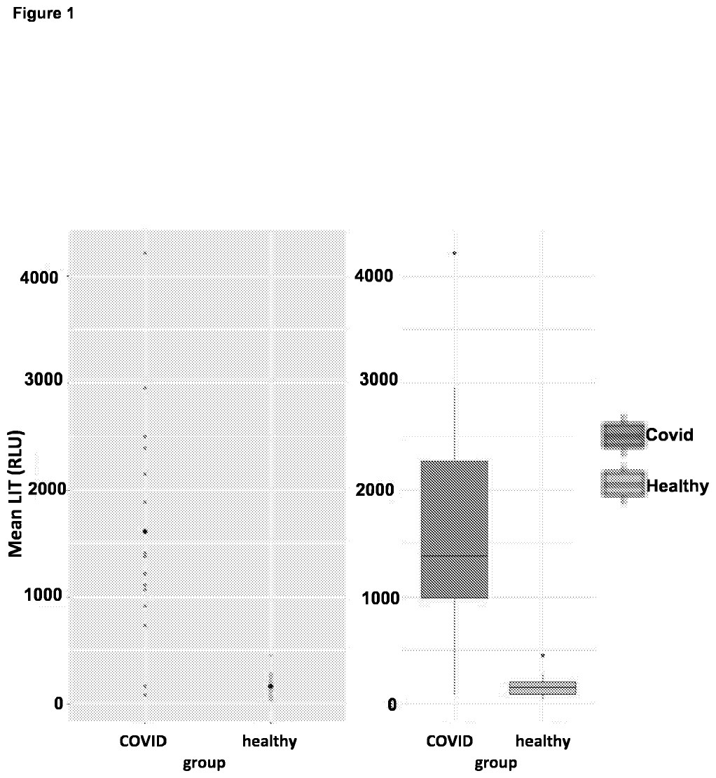

Scores for neutrophil functionality level for each individual in these categories were averaged (mean) and these averages plotted as shown in FIG. 1.

### Results

The data shows strong significant difference in LIT score between the two cohorts with the “COVID” group having a far greater mean LIT score compared with the “healthy volunteers” cohort.

TABLE 1
Mean (SD) and median (IQR) values
for COVID and volunteer groups

| Group | N | Mean ± SD | Median (IQR) |
|---|---|---|---|
| healthy | 18 | 167 ± 99 | 153 (112) |
| COVID | 15 | 1614 ± 1096 | 1378 (1276) |

The median LIT score for patients with COVID-19 was 9-fold higher than values within the control healthy blood donor group (Mann-Whitney U test, p<<<0.05)

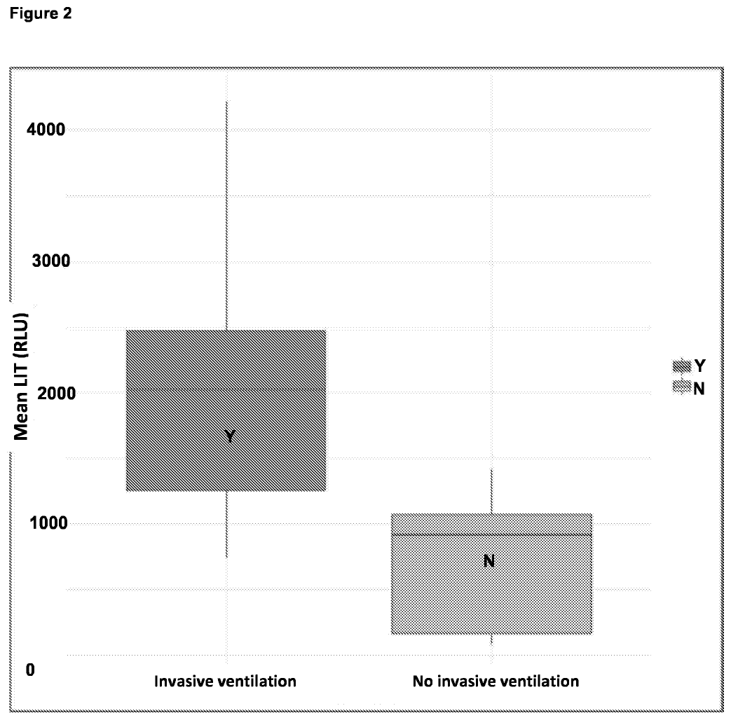

10 patients with COVID-19 were undergoing mechanical ventilation at the time of sampling. Median LIT values were over 2-fold elevated in this group compared to non-ventilated patients (2020 vs 916 respectively; Mann Whitney U test, p=0.02). See FIG. 2.

Mean LIT scores for individuals were also compared with white blood cell (WBC) count and neutrophil count. LIT scores were found to correlate strongly with both counts. Correlation with WBC was 0.73 (Spearman's [0.39, 0.90], p=0.003) with a somewhat higher value of 0.80 for the neutrophil count (Spearman's [0.52,0.92], p=0.0004).

These results for the first time demonstrate that neutrophil functionality level as assessed by LIT score can be used as an indicative measure of the onset of severe COVID-19 disease and as a tool to monitor disease progression, interventions and recovery. LIT scores may for example be used to provide rapid indication of severity meriting need for admission to an ICU, e.g. onset or occurrence of severity of at least 5 (warranting non-invasive ventilation or high flow oxygen) or 6 (warranting mechanical ventilation) on the WHO Ordinal Scale for Clinical Improvement for COVID-19 disease. LIT scores may also be used to conveniently monitor treatment interventions in such patients

### Example 3: Comparison of LIT Scores of COVID-19 Patients and Non-COVID-19 Sepsis Patients

Blood samples from 12 ICU patients with non-COVID-19 related sepsis were also analysed for LIT score in the same manner as the blood samples from COVID-19 patients as discussed above. The median LIT score within this patient cohort was 2120 (IQR: 2024), 14-fold higher than the median value of 153 in the volunteer healthy cohort (Table 1; Mann-Whitney U test, p<0.05). Thus, higher ROS production was observed than in the COVID-19 patient cohort. No association was found between LIT score and the use of invasive ventilation within the sepsis patient group (Mann-Whitney U test, p=0.88)

TABLE 2
Mean (SD) and median (IQR) values
for sepsis and volunteer groups

| Group | N | Mean ± SD | Median (IQR) |
|---|---|---|---|
| healthy | 18 | 167 ± 99 | 153 (112) |
| sepsis | 12 | 2526 ± 1616 | 2120 (2024) |

As with the COVID-19 patients, LIT scores correlated strongly with the peripheral neutrophil count (R=0.76 [018,1.00], p=0.009).

Thus LIT score is also suggested as a potential tool to monitor sepsis progression and treatment management and preferable to merely counting neutrophil number. Higher ROS production than in healthy controls can be expected to activate a range of downstream processes that may contribute to tissue damage (Brinkmann et al. (2010); Naik and Dixit (2011); Sheshachalam et al. (2014); Miralda et al. (2017)).

### Example 4: Use of LIT Score Relative to Neutrophil Count

As shown in FIG. 3, LIT score was plotted against neutrophil count in the blood samples of the COVID-19 and sepsis patient cohorts with the neutrophil count being expressed as neutrophils×10⁹/l. The gradient of this line was taken to determine the LIT-N value which represented the increase in LIT for each increase of 1×10⁹/l in the blood neutrophil count. Median LIT-N values for patients with COVID-19 and non-COVID sepsis were 152 and 115 respectively indicating that relative ROS production from neutrophils was higher in COVID-19 patients.

LIT-N scores were also looked at in relation to the 28-day mortality of patients on the ICU. 4 out of the 26 patients died within this period of which 2 succumbed within 24 hours of the LIT test. One of these patients had COVID-19 infection. The personal LIT-N value was markedly low at 9 compared to the median of 152 for the whole COVID-19 patient group. The second patient had non-COVID sepsis. In this case, the LIT-N score was markedly elevated in the period prior to death at 419. The other two patients had LIT-N scores similar to the median of their group but death did not occur until 4 and 26 days respectively after testing.

On the basis of these preliminary studies, LIT-N score as a measure of extreme hypo- or hyperfunctional neutrophil activity was further investigated as a means of providing a biomarker of markedly increased risk of short-term mortality; see Example 5 below. LIT-N scores are proposed as having considerable value in clinical management of both severely ill COVID-19 and sepsis patients.

Current laboratory assays of immune cell function rely almost exclusively on measurement of cell number (Urrechaga 2020). In summary, the results now provided indicate that LIT-N scores (challenge-induced increase of superoxide production above basal per 10⁹ neutrophils/l) may provide a more useful manner to assess severity of disease in both COVID-19 and sepsis patients, and indeed patients with other severe infection, which focusses on the functional capacity of neutrophils.

### Example 5: Use of LIT Scores and Neutrophil Count for Assessing Mortality Risk in Hospitalised COVID-19 Patients in an ICU

LIT/N scores [calculated from challenge-induced superoxide production determined as above and neutrophil count expressed as neutrophils×10⁹/l (N)] were compared for blood samples of 44 COVID-19 patients admitted to an ICU in Turkey. Blood samples were taken daily from ICU admission to either discharge (n=24) or death (n=20). Mathematical modelling (Linear Mixed Effects Model) was used to project the trajectory of neutrophil count alone or LIT/N (LIT score divided by neutrophil count). As shown by FIG. 4, it was found that neutrophil count alone does not provide sufficient mortality prediction capability. In contrast, it was found that by further employing LIT scores for challenge-induced superoxide production and converting these to LIT/N scores (challenge-induced increase of superoxide production above basal per 10⁹ neutrophils/l), a far better predictor of mortality risk was attained when such scores were compared with patient survival or death; see FIG. 5. As shown by FIG. 5, predicted values of LIT/N were 0.15 or above for all patients admitted to ICU but showed a downward trend for survivors.

### REFERENCES

Arcanjo et al. (2020) The emerging role of neutrophil extracellular traps in severe acute respiratory syndrome coronavirus 2 (COVID-19). Sci. Rep. 10, 19630
Barnes et al. (2020) Targeting potential drivers of COVID-19: neutrophil extracellular traps. J. Exp. Med. 217: e2020065
Brinkmann et al. (2010). Neutrophil extracellular traps: how to generate and visualize them. J. Vis. Exp. e1724. 10.3791/1724
Cavalcante-Silva et al. (2021; on-line 30-11-2020). Neutrophils and COVID-19: The road so far. Int. Immunopharmacol. 90:107233.
Cecchini R. & Cecchini A. L. (2020) SARS-CoV-2 infection pathogenesis is related to oxidative stress as a response to aggression. Med. Hypotheses. 143, 110102
Chan AS & Rout A. Use of Neutrophil-to-Lymphocyte and Platelet-to-Lymphocyte Ratios in COVID-19. J. Clin. Med. Res. 12 (7), 448-53.
Chen et al. (2020) Epidemiological and Clinical characteristics of 99 cases of 2019 novel coronavirus pneumonia in Wuhan, China: a descriptive study. Lancet 395 (10223); 507-13.
Dhama et al. (2020) Coronavirus disease 2019-COVID-19. Clin. Microbiol. Rev. 33 (4)
Du et al. (2020). Predictors of mortality for patients with COVID-19 pneumonia caused by SARS-CoV-2: a prospective cohort study. Eur. Respir. J. (2020) 55: e2000524
Feng et al. (2020) Severe acute respiratory syndrome coronavirus 2 (SARS-CoV-2): a review. Mol Cancer 19 (1): 100.
Fox et al. (2020) Pulmonary and cardiac pathology in Covid-19: the first autopsy series from New Orleans. Lancet Respir Med. 8, 681-6
Fullerton et al. (2019) Repurposing the Oxford MediStress Leukocyte Coping Capacity™ assay as a novel point-of-care biomarker of neutrophil function. Presentation British Pharmacological Society Edinburgh, U K 2019
Galani I. E & Andreakos E. (2015) Neutrophils in viral infections: Current concepts and caveats. J. Leukoc. Biol. 98, 557-564.
Ghahramani et al. (2020) Laboratory features of severe vs. non-severe COVID-19 patients in Asian populations: a systematic review and meta-analysis. Eur. J. Med. Res. 25:30
Golonka et al. (2020). Harnessing innate immunity to eliminate SARS-CoV-2 and ameliorate COVID-19 disease. Physiol. Genomics 52, 217-221.
Hellebrekers et al. (2018) Neutrophil phenotypes in health and disease. Eur. J. Clin. Invest. 48, e12943
Hoffmann et al. (2020) SARS-CoV-2 Cell Entry Depends on ACE2 and TMPRSS2 and Is Blocked by a Clinically Proven Protease Inhibitor. Cell 181:271-280
Huang et al. (2020) Clinical features of patients infected with 2019 novel coronavirus in Wuhan, China. Lancet 395, 497-506
Hu et al. (1999) Intracellular free calcium regulates the onset of the respiratory burst of human neutrophils activated by phorbol myristate acetate, Cell Signal 11, 335-360
Karlsson-Bengtsson et al. (2000). Phorbol myristate acetate induces neutrophil NADPH-oxidase activity by two separate signal transduction pathways: Dependent or independent of phosphatidylinositol 3-kinase. J. Leukocyte Biol. 67, 396-404.
Kruger et al. (2015). Neutrophils: Between host defence, immune modulation, and tissue injury. PLOS Pathog. 11: e1004651. 10.1371
Laforge et al. (2020) Tissue damage from neutrophil-induced oxidative stress in COVID-19 Nat. Rev. Immunol. 20, 515-516
Lagunas-Rangel F. A. (2020) Neutrophil-to-lymphocyte ratio and lymphocyte-to-C-reactive protein ratio in patients with severe coronavirus disease 2019 (COVID-19): A meta-analysis. J. Med. Virol. 92:1733-1734.
Lambeth J. D. (2004) NOX enzymes and the biology of reactive oxygen. Nat. Rev. Immunol. 4, 181-189.
Lan. et al. Structure of the SARS-CoV-2 spike receptor-binding domain bound to the ACE2 receptor. Nature 581, 215-220
Li et al. (2020) Predictive values of neutrophil-to-lymphocyte ratio on disease severity and mortality in COVID-19 patients: a systematic review and meta-analysis. Crit. Care 24, 647
Li et al. (2003) Angiotensin-converting enzyme 2 is a functional receptor for the SARS coronavirus. Nature 426:450-4
Liu et al. (2020) Neutrophil-to-lymphocyte ratio as an independent risk factor for mortality in hospitalized patients with COVID-19. J. Infect. 81: e6-e12
Lucas et al. (2020) Longitudinal analyses reveal immunological misfiring in severe COVID-19. Nature 584:463.
McLaren et al. (2003) Leukocyte coping capacity: A novel technique for measuring the stress response in vertebrates. Exp. Physiol. 88 (4): 541-6
Mian et al. (2005) Stress a radical approach to old problems in Oxington K. editor, Stress and health: New Research. NY USA: Novo Science: p 61-79
Miralda et al. (2017). Multiple phenotypic changes define neutrophil priming. Front. Cell. Infect. Microbiol. 7:217.
Mocsai A. (2013). Diverse novel functions of neutrophils in immunity, inflammation, and beyond. J. Exp. Med. 210, 1283-1299.
Mocsai et al. (2006). Integrin signaling in neutrophils and macrophages uses adaptors containing immunoreceptor tyrosine-based activation motifts. Nat. Immunol. 7, 1326-13
Mocsai et al. (2011). The SYK tyrosine kinase: a crucial player in diverse biological functions. Nat. Rev. Immunol. 10, 387-402.
Mochas et al. (2002). Syk is required for integrin signaling in neutrophils. Immunity 16, 547-558
Mohamed et al. (2020) Neutrophil elastase inhibitors: A potential prophylactic treatment option for SARS-CoV-2-induced respiratory complications? Crit. Care. 24:31
Montas et al. (2011) Radical health: ecological corollaries of body condition, transport stress and season on plasma antioxidant capacity in the European badger, J. Zoology 284 (2) 114-123.
Mortaz et al. (2020) The Immune Response and Immunopathology of Covid-19. Front. Immunol. 11:2037
Naik E. & Dixit V. M. (2011). Mitochondrial reactive oxygen species drive proinflammatory cytokine production. J. Exp. Med. 208, 417-420.
Naumenko et al. (2018) Neutrophils in viral infection. Cell Tissue Res. 371, 505-516.
Nathan C. & Cunningham-Bussel A. (2013). Beyond oxidative stress: an immunologist's guide to reactive oxygen species. Nat. Rev. Immunol. 13, 349-361
Nguyen et al. (2017). Neutrophils to the ROScue: Mechanisms of NADPH Oxidase Activation and Bacterial Resistance. Frontiers Cell Infect. Microbiol. 7:373
Panday et al. (2015) NADPH oxidases: an overview from structure to innate immunity-associated pathologies. Cell Mol. Immunol. 12, 5-23
Parackova et al. (2020) Disharmonic Inflammatory Signatures in COVID-19: Augmented Neutrophils' but Impaired Monocytes' and Dendritic Cells' Responsiveness. Cells 9:2206
Petito et al. (2020). Association of Neutrophil Activation, More Than Platelet Activation With Thrombotic Complications in Coronavirus Disease 2019 J. Infect. Diseases, jiaa756
Ponti et al. (2020). Biomarkers associated with COVID-19 disease progression. Crit. Rev. Clin. Lab. Sci. 57:389-399.
Roda et al. (2014) Integrating Bioluminescence Detection on Smartphones: Mobile Chemistry Platform for Point-of-Need Analysis. Anal. Chem. 86 (15), 7299-7304
Schulte-Schrepping et al. (2020) Severe COVID-19 is marked by a dysregulated myeloid cell compartment. Cell. 182, 1419-1440
Selders et al. (2017) An overview of the role of neutrophils in innate immunity, inflammation and host-biomaterial integration. Regen. Biomater. 4, 55-68
Shelton-Rayner et al. (2010), Leukocyte reactivity as an objective means of quantifying mental loading during ergonomic evaluation, Cell. Immunol. 263, 1, (22-30)
Shelton-Rayner et al. (2011) Quantitative Physiological Assessment of Stress Via Altered Immune Functioning Following Interaction With Differing Automotive Interface Technologies, Int. J. Human-Computer Interaction, 27, 9, 900-919
Shelton-Rayner et al. (2012) Leukocyte responsiveness, a quantitative assay for subjective mental workload, Int. J. Industrial Ergonomics 42, 1, 25-33.
Shelton-Rayner et al. (2011) Quantifying Transient Psychological Stress Using a Novel Technique: Changes to PMA-Induced Leukocyte Production of ROS In Vitro, Int, J. Occupational Safety and Ergonomics, 17:1, 3-13
Shelton-Rayner et al. (2011) Quantitative Physiological Assessment of Stress Via Altered Immune Functioning Following Interaction With Differing Automotive Interface Technologies, Int. J. Human-Computer Interaction, 27:9, 900-919,
Sheshachalam et al. (2014). Granule protein processing and regulated secretion in neutrophils. Front. Immunol. 5:448.
Soehnlein et al. (2017). Neutrophils as protagonists and targets in chronic inflammation. Nat. Rev. Immunol. 17:248-261.
Tabata et al. (2020) Clinical characteristics of COVID-19 in 104 people with SARS-CoV-2 infection on the Diamond Princess cruise ship: a retrospective analysis. Lancet Infect. Dis. 20:1043.
Teuwen, et al. (2020) COVID-19: the vasculature unleashed. Nat. Rev. Immunol. 20, 389-391
Urrechaga, E. (2020) Reviewing the value of leuckocyte cell population date (CPD) in the management of sepsis. Ann. Transl. Med. 15:953
Wang et al. (2020) Clinical Characteristics of 138 Hospitalized Patients With 2019 Novel coronavirus-infected pneumonia in Wuhan, China. JAMA 323, 1061-1069.
Wu et al. (2020). Estimating clinical severity of COVID-19 from the transmission dynamics in Wuhan, China. Nat. Med. 26, 506-510
Yang et al. (2020) COVID-19: immunopathogenesis and immunotherapeutics. Signal Transduction Targeted Therapy 5, no. 128
Yao et al. (2020) A pathological report of three COVID-19 cases by minimally invasive autopsies. 49, 411-417
Ye et al. (2020). Dynamic changes of D-dimer and neutrophil-lymphocyte count ratio as prognostic biomarkers in COVID-19. Respir. Res. 21:169.
Zeng et al. (2020) Can we predict the severity of coronavirus disease 2019 with a routine blood test? Pol. Arch. Intern. Med. 130 (5): 400-6.
Zhang et al. (2020) Structure of the SARS-CoV-2 spike receptor-binding domain bound to the ACE2 receptor. Nature 581:215-220.

1. A method of assessing disease progression in a subject suspected or known to have a viral infection capable of causing acute respiratory disease syndrome (ARDS), which comprises:
(a) contacting a whole blood sample obtained from said subject with an inducer capable of stimulating superoxide production in neutrophils under conditions suitable for such stimulation;
(b) determining the increase of superoxide production above basal in said test sample after a time period to obtain a first result and
(c) comparing said first result with a second comparator result, wherein said second comparator result is derived from carrying out steps (a) and (b) with blood samples from healthy individuals or is a pre-determined threshold which correlates with one or more criteria equating with onset or occurrence of an ARDS disease status, whereby ARDS disease status is determined.

2. A method of monitoring treatment in a subject known to have disease arising from a viral infection capable of causing ARDS which comprises:
(a) contacting a whole blood sample obtained from said subject with an inducer capable of stimulating superoxide production in neutrophils under conditions suitable for such stimulation;
(b) determining the increase of superoxide production above basal in said test sample after a time period to obtain a first result and
(c) comparing said first result with a second comparator result which has been taken from the subject at an earlier time point at the start or during treatment, whereby reduction in induced superoxide production in the test sample compared with in said second comparator sample is indicative of reduction in disease severity.

3. A method as claimed in claim 1 wherein in step (b) neutrophil count in the test sample is also determined.

4. A method as claimed in claim 3 wherein in step (b) the increase of superoxide production above basal is determined per 109 neutrophils/1 to obtain a LIT/N score.

5. A method as claimed in claim 1 wherein said subject is suspected or known to be infected with a coronavirus.

6. A method as claimed in claim 5 wherein said coronavirus is a SARS virus capable of causing severe acute respiratory syndrome.

7. A method as claimed in claim 6 wherein said subject is suspected or known to be infected with SARS-CoV-2 capable of giving rise to COVID-19 disease.

8. A method as claimed in claim 1 for assessing disease progression wherein in step (c) said first result is compared with a second comparator result representing a pre-determined threshold for onset of severity warranting hospitalisation and/or provision of oxygen.

9. A method as claimed in claim 8 wherein the subject is suspected or known to have COVID-19 and in step (c) said first result is compared with a second comparator result representing a pre-determined threshold for onset or occurrence of severity of at least 3 (warranting at least hospitalisation), or at least 4 (warranting provision of oxygen) or at least 5 (warranting non-invasive ventilation or high flow oxygen) or at least 6 (warranting intubation and mechanical ventilation) on the WHO Ordinal Scale for Clinical Improvement for COVID-19 disease.

10. A method as claimed in claim 4 which further comprises determining LIT/N score at more than one time point to assess mortality risk, for example wherein LIT/N score is determined at more than one time point, e.g. daily over two or more days, in a patient with severe COVID-19 (at least 4 or 5 on the WHO Ordinal Scale) to assess mortality risk.

11. A method as claimed in claim 4 wherein LIT and neutrophil count scores of the subject are mapped to a LIT vs neutrophil count plot to determine the subject's LIT-N™ status as a means of assessing mortality risk.

12. A method as claimed in claim 1, wherein the inducer capable of stimulating superoxide production in neutrophils is phorbol myristate acetate (PMA), N-Formyl-Met-Leu-Phe (fMLP chemotactic peptide), zymosan, lipopolysaccharide or adrenaline.

13. A method as claimed in claim 12 wherein the inducer is PMA.

14. A method as claimed in claim 1 wherein superoxide production is detected by chemiluminescence detection.

15. A method as claimed in claim 14, wherein superoxide production is detected using luminol or isoluminol and the resulting chemiluminescence is measured.

16. A method as claimed in claim 15, wherein the inducer capable of stimulating superoxide production in neutrophils is phorbol myristate acetate (PMA), superoxide production is detected using luminol as an amplifier and the resulting chemiluminescence is measured.

17. A system specifically configured for carrying out a method according to claim 14, said system comprising a photon detector for quantitative detection of chemiluminescence and a system for analysing the results and configured to provide an alert for a neutrophil functionality level associated with a pre-determined threshold correlating with a disease status, preferably wherein said photon detector is a portable luminometer.
